(19) United States
(12) Patent Application Publication
(10) Pub. No.: US 2024/0398752 A1
(43) Pub. Date: Dec. 5, 2024

(54) PHARMACEUTICAL COMPOSITION FOR PREVENTING OR TREATING LIVER CIRRHOSIS COMPRISING ISOSAKURANETIN OR PHARMACEUTICALLY ACCEPTABLE SALT THEREOF AS ACTIVE INGREDIENT

(71) Applicant: AJOU UNIVERSITY INDUSTRY-ACADEMIC COOPERATION FOUNDATION, Suwon-si, Gyeonggi-do (KR)

(72) Inventors: So Hee Kim, Seoul (KR); Hyoungsu Kim, Suwon-si, Gyeonggi-do (KR); Sunghun Bae, Daegu (KR)

(73) Assignee: AJOU UNIVERSITY INDUSTRY-ACADEMIC COOPERATION FOUNDATION, Suwon-si, Gyeonggi-do (KR)

(21) Appl. No.: 18/698,383

(22) PCT Filed: Oct. 21, 2022
(86) PCT No.: PCT/KR2022/016128

§ 371 Date: Apr. 4, 2024

(30) Foreign Application Priority Data
Oct. 21, 2021 (KR) 10-2021-0141173

(51) Int. Cl. A61K 31/352; A61K 9/00; A61P 1/16

(52) U.S. Cl. CPC A61K 31/352; A61K 9/0056; A61P 1/16

(57) ABSTRACT

The present invention relates to a pharmaceutical composition for preventing or treating liver cirrhosis comprising Isosakuranetin or a pharmaceutically acceptable salt thereof as an active ingredient, wherein the pharmaceutical composition can inhibit the death of hepatocytes and suppress the increase in GOT, GPT, ALP, or total cholesterol as liver cirrhosis or hepatic fibrosis cells are treated with Isosakuranetin.

### TECHNICAL FIELD

The present disclosure relates to a pharmaceutical composition for preventing or treating liver cirrhosis, including isosakuranetin or a pharmaceutically acceptable salt thereof as an active ingredient.

### BACKGROUND ART

The liver is a type of organ that performs various functions, and in recent years, the incidence of liver disease and death due to the liver disease have been gradually increasing. A trend of liver disease in Korea is that, although there were many patients with hepatitis B or C in the past, now, as in Europe and the United States, the number of patients with diseases caused by alcohol is increasing over the hepatitis-related liver diseases, and the number of female patients tends to increase compared to the past due to increased drinking experiences alongside increased social activities of women. In line with this trend, research on therapeutic agents for the liver diseases is actively underway around the world.

In general, liver cirrhosis is the final condition of chronic liver disease. The main causes of liver cirrhosis are hepatitis virus infection, alcoholism, impaired secretion of bile acid, drug addiction, allergies, and excessive accumulation of iron.

Hepatocytes have strong regenerability and may be repaired by regeneration even if they are destructed to a degree, but at some point, they become fibrous without regeneration, and the liver hardens after fibrosis. This change in the structure of the liver and hardening of the liver so that it cannot return to its original state is called liver cirrhosis.

The process of liver fibrosis, which leads to liver cirrhosis, is a response to sustained stimuli and may be, similar to the wound healing process, divided into three phases including acute inflammation, synthesis of collagen and components constituting the extracellular matrix (ECM), and tissue reorganization (scar formation). After the destruction of hepatocytes, in the process of tissue reorganization into new hepatocytes, complete cell structures such as the cytoplasm and extracellular membrane are not formed, and only the extracellular matrix that maintains the skeletal structure of the cell is developed to cause excessive accumulation of collagen, which is the main component of the extracellular matrix of hepatocytes, resulting in liver cirrhosis as the liver tissue hardens due to fibrous collagen.

Liver fibrosis is a disease in which the liver tissue in a chronic inflammatory state is repeatedly damaged and regenerated to cause connective tissues such as collagen in the tissue to be deposited excessively and form scars in the liver tissue.

In general, liver fibrosis, unlike hepatocirrhosis, is reversible and consists of thin fibrils without formation of nodules. In addition, the loss of factors that cause liver damage would make the normal recovery possible. However, if this mechanism of liver fibrosis persists repeatedly, it progresses to irreversible liver cirrhosis, which is characterized by increased crosslinking between connective tissues, the accumulation of thick fibrils, and a loss of the normal structure of the hepatic lobules to form nodules.

Liver diseases have various causes, but when it becomes chronic, it eventually leads to liver fibrosis or cirrhosis, regardless of the cause. The absence of initial subjective symptoms makes liver diseases difficult to diagnose early, and detection is generally made in the chronic state, causing social problems due to difficulty in treatment and high mortality rate. In addition, no therapeutic agent with high efficacy has yet been developed.

### DISCLOSURE OF THE INVENTION

### Technical Goals

An object of the present disclosure is to provide a composition for preventing or treating liver cirrhosis, including isosakuranetin or a pharmaceutically acceptable salt thereof as an active ingredient.

### Technical Solutions

To achieve the above object, the present disclosure provides a pharmaceutical composition for preventing or treating liver cirrhosis, including isosakuranetin or a pharmaceutically acceptable salt thereof as an active ingredient.

Further, the present disclosure provides a health functional food composition for preventing or ameliorating liver cirrhosis, including isosakuranetin as an active ingredient.

Furthermore, the present disclosure provides a health functional food composition that helps liver health, including isosakuranetin as an active ingredient.

### Advantageous Effects

According to the present disclosure, treatment of isosakuranetin to rats with liver damage induced shows an effect of preventing liver cirrhosis by inhibiting progression of liver damage in a concentration-dependent manner so as to protect the liver, so thus functions of the liver and kidney may be improved.

### BRIEF DESCRIPTION OF DRAWINGS

FIG. 1 shows graphs of changes in body weight and weight of liver and kidney depending on a concentration of isosakuranetin in rats with liver damage induced by DMN.

FIG. 2 shows graphs of evaluating hepatic functional activities depending on a concentration of isosakuranetin in rats with liver damage induced by DMN.

FIG. 3 shows graphs of evaluating renal functional activities depending on a concentration of isosakuranetin in rats with liver damage induced by DMN.

FIG. 4 shows images of evaluating histological changes in the liver and kidney depending on a concentration of isosakuranetin in rats with liver damage induced by DMN.

FIG. 5 shows curve graphs of a mean plasma concentration after intravenous (10 mg/kg) and oral (20 mg/kg) administration of tofacitinib to CON, LC, and LC-ISN rats to identify a hepatoprotective effect of isosacuranetin in the in-vivo pharmacokinetic experiments: (A) a mean plasma concentration curve of tofacitinib after intravenous administration of 10 mg/kg of tofacitinib to CON (black; n=5), LC (red; n=6), and LC-ISN (blue; n=7) rats, and (B) a mean plasma concentration curve of tofacitinib after oral administration of 20 mg/kg of tofacitinib to CON (black; n=6), LC (red; n=7), and LC-ISN (blue; n=7) rats.

FIG. 6 shows graphs of Vₘₐₓ, Kₘ, and CLᵢₙₜ values measured by the in-vitro experiment for enzyme activity using small intestinal and hepatic microsomes of CON, LC, and LC-ISN groups to identify a hepatoprotective effect of isosakuranetin in the in-vitro experiment for enzyme activity: mean values of Vₘₐₓ, Kₘ, and CLᵢₙₜ in microsomes of the liver (A) and small intestine (B) of CON, LC, and LC-ISN groups. Vₘₐₓ, a maximum rate of drug metabolism; Kₘ, a concentration of a drug at 50% rate of Vₘₐₓ; CLᵢₙₜ, intrinsic clearance (**p<0.01, ***p<0.001).

FIG. 7 shows experimental results of immunoblot analysis to identify a drug metabolizing enzyme recovery effect of isosakuranetin, illustrating protein expression of CYP isoforms, P-gp, CAR, and PXR in the small intestinal and hepatic microsomes of CON, LC, and LC-ISN rat groups.

### BEST MODE FOR CARRYING OUT THE INVENTION

Hereinafter, in order to describe the present disclosure more specifically, preferred example embodiments according to the present disclosure will be described in detail with reference to the accompanied drawings. However, the present disclosure is not limited to the example embodiments described herein and may be implemented in other forms.

The present disclosure provides a pharmaceutical composition for preventing or treating liver cirrhosis, including isosakuranetin or a pharmaceutically acceptable salt thereof as an active ingredient.

The isosakuranetin is a compound represented by the following Chemical Formula 1 and a type of flavanone.

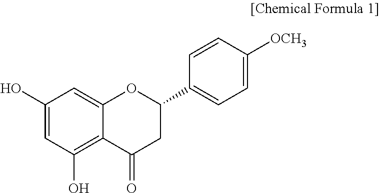 [SMILES: COc1ccc([C@@H]2CC(=O)c3c(O)cc(O)cc3O2)cc1]

The isosakuranetin has a molecular formula represented by C16H14O5 with a molecular weight of 286.3 and is also named 4′-methylnaringenin or (2S)-5,7-dihydroxy-2-(4-methoxyphenyl)-2,3-dihydrochromen-4-one. It may be extracted with solvents such as chloroform, dichloromethane, ethyl acetate, DMSO, and acetone.

The isosakuranetin has hepatoprotective activity and renal function-enhancing activity.

Specifically, the isosakuranetin may inhibit tissue damage including necrosis and fibrosis in liver and inhibit tissue damage accompanied by massive cell death in kidney. More specifically, it may inhibit activities of GOT, GPT, or ALP to promote the breakdown of total cholesterol and stimulate synthesis of total protein and albumin.

In addition, the isosakuranetin may restore drug metabolizing enzymes reduced by liver cirrhosis, wherein the drug metabolizing enzymes may be one or more types selected from the group consisting of CYP2C11 and CYP3A1/2.

The isosakuranetin may be included in the pharmaceutical composition at a concentration of 3 to 20 mM or 4 to 15 mM.

The isosakuranetin may be administered in an amount of 1 to 20 mg/kg, 5 to 20 mg/kg, 5 to 15 mg/kg, or 10 to 15 mg/kg of body weight. It may be contained in the above concentration to suppress liver damage and enhance hepatoprotective activity. Specifically, 4 mL of isosakuranetin may be administered per kilogram of the body weight.

The pharmaceutical composition may additionally include carriers, excipients, or diluents commonly used in preparation of pharmaceutical compositions.

If the composition of the present disclosure is a pharmaceutical composition, for administration, it may include pharmaceutically acceptable carriers, excipients, or diluents in addition to the active ingredient described above. The carrier, excipient, and diluent may include lactose, dextrose, sucrose, sorbitol, mannitol, xylitol, erythritol, maltitol, starch, gum acacia, alginate, gelatin, calcium phosphate, calcium silicate, cellulose, methyl cellulose, microcrystalline cellulose, polyvinylpyrrolidone, water, methylhydroxybenzoate, propylhydroxybenzoate, talc, magnesium stearate, and mineral oil.

Each of the pharmaceutical compositions of the present disclosure may be used by being formulated in the form of oral formulations, such as acids, granules, tablets, capsules, suspensions, emulsions, syrups, and aerosols, topical agents, suppositories, or sterile injection solutions according to the conventional method. Specifically, when formulated, it may be prepared using diluents or excipients such as fillers, weighting agents, binders, wetting agents, disintegrating agents, and surfactants that are conventionally used. Solid preparations for oral administration include tablets, pills, acids, granules, and capsules, but are not limited thereto. Such solid preparations may be prepared by mixing, in addition to the above active ingredients, at least one or more excipients such as starch, calcium carbonate, sucrose, lactose, and gelatin. In addition to simple excipients, lubricants such as magnesium stearate and talc may also be used. In addition to liquid substances and liquid paraffin for oral use, various excipients, such as wetting agents, sweeteners, aromatics, and preservatives may be added for preparation. Preparations for parenteral administration include sterilized aqueous solutions, non-aqueous solvents, suspensions, emulsions, lyophilized preparations, and suppositories. As non-aqueous solvents and suspensions, propylene glycol, polyethylene glycol, vegetable oils such as olive oil, and injectable ester such as ethyl oleate may be used. As a base of suppositories, Witepsol, Macrosol, Tween 61, cacao butter, laurin fat, and glycerogelatin may be used.

The pharmaceutical composition of the present disclosure may be administered orally or parenterally (e.g., intravenously, subcutaneously, intraperitoneally, or topically) depending on a desired method, preferably orally. In addition, the dosage of the pharmaceutical composition of the present disclosure varies depending on the subject's weight, age, sex, health status, diet, duration of administration, method of administration, excretion rate, and severity of the disease, but is not limited thereto.

The pharmaceutical composition may be formulated in the form of one or more external preparations selected from the group consisting of creams, gels, patches, sprays, ointments, emplastrum agents, lotions, liniments, pastas, and cataplasmas.

In addition, the present disclosure provides a health functional food composition for preventing or ameliorating liver cirrhosis, including isosakuranetin as an active ingredient. Alternatively, the present disclosure provides a health functional food composition that supports liver health, including isosakuranetin as an active ingredient.

The isosakuranetin is a compound represented by the following Chemical Formula 1 and a type of flavanone.

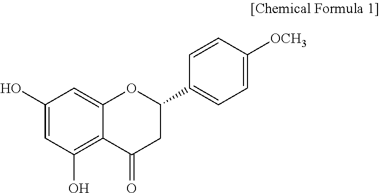 [SMILES: COc1ccc([C@@H]2CC(=O)c3c(O)cc(O)cc3O2)cc1]

The isosakuranetin has a molecular formula represented by C16H1405 with a molecular weight of 286.3 and is also named 4′-methylnaringenin or (2S)-5,7-dihydroxy-2-(4-methoxyphenyl)-2,3-dihydrochromen-4-one. It may be extracted with solvents such as chloroform, dichloromethane, ethyl acetate, DMSO, and acetone.

Corresponding features may be substituted for the above.

The present disclosure may be generally used as a commonly used food product.

As used herein, the term “health functional food” refers to food manufactured and processed using raw materials or components with functionality useful for the human body and also means food that are processed to efficiently have bioregulatory functions in addition to nutritional supply so as to derive high medicinal and clinical effects, and may be used interchangeably with terms that are known in the art such as those used in functional food. For example, it may be prepared as dietary supplements, food for special nutritional supplements, and functional beverages, or the composition according to the present disclosure may be added in natural food and processed food.

The health functional food composition may be prepared as powder, granule, tablet, capsule, syrup, or beverage, with no limitation in the form that the health functional food composition may take, and it may include all foods in the ordinary sense. For example, it may include beverages and various drinks, fruits and processed foods thereof (canned fruits, jams, etc.), fish, meat and processed foods thereof (ham, bacon, etc.), breads and noodles, cookies and snacks, and dairy products (butter, cheese, etc.), and all functional foods in the usual sense may be included. It may also include foods that are used as feed for animals.

The health functional food composition according to the present disclosure may be prepared by further including foodologically acceptable food additives and other appropriate supplementary ingredients commonly used in the art. For example, it may further include, for example, flavors, natural carbohydrates, sweeteners, vitamins, electrolytes, colorants, pectic acids, alginic acids, organic acids, protective colloidal thickeners, pH regulators, stabilizers, preservatives, glycerin, alcohols, and carbonators. In particular, monosaccharides such as glucose and fructose, disaccharides such as maltose and sucrose, and polysaccharides such as dextrin and cyclodextrin, sugar alcohols such as xylitol, sorbitol, and erythritol may be used as the natural carbohydrates, and natural sweeteners such as thaumatin and stevia extract or synthetic sweeteners such as saccharin and aspartame may be used as sweeteners. It is preferred that the food additives and other auxiliary ingredients are used by being added in trace amounts as long as the purpose to which they are added may be achieved. The trace amount, when expressed numerically, may range from 0.0005 wt % to about 0.5 wt % based on the total weight of the health functional food composition.

The health functional food composition according to the present disclosure has the advantage of not causing side effects that may occur in the long-term intake of drugs by using food as a raw material, unlike ordinary drugs, while securing excellent portability, such that it may be taken as an adjuvant for the darkening of gray hair or protection from photodamage.

### MODES FOR CARRYING OUT THE INVENTION

Hereinafter, example embodiments will be described in detail to help understanding of the present disclosure. However, the following example embodiments are merely illustrative of the content of the present disclosure, and the scope of the present disclosure is not limited to the following example embodiments. The example embodiments of the present disclosure are provided to more completely explain the present disclosure to those with average skill in the art.

### Experimental Example Experimental Materials and Methods

The following Experimental Examples are intended to provide experimental examples that are commonly applied to each Example according to the present disclosure.

### 1. Materials

Isosakuranetin used in this experiment was purchased from Sigma-Aldrich.

The laboratory rats used in this experiment were administered with 10 mg/kg of N-dimethylnitrosamine (DMN, dissolved in saline) intraperitoneally for 3 consecutive days every week for 6 weeks (e.g., every Monday, Tuesday, and Wednesday for 6 weeks) to induce liver cirrhosis.

### 2. Experiment for Prophylactic and Therapeutic Effects of Liver Cirrhosis

In order to determine whether there is a hepatoprotective effect, for normal rats (control, CON), rats with liver cirrhosis induced by DMN, and rats with liver cirrhosis induced and administered orally with 5 mg/kg (ISN 5), 10 mg/kg (ISN 10), and 15 mg/kg (ISN 15) of isosakuranetin, respectively, every day for 6 weeks, the hepatic and renal functions of the rats were measured.

For example, changes in the body weight, liver weight, and kidney weight of rats were measured, and indicators to evaluate the hepatic functional activity and renal functional activity of rats are shown in Table 1.

### 3. Changes in Weight

The changes in the body weight, liver weight, and kidney weight of laboratory rats were measured.

### 4. Changes in Hepatic Functional Activities

Measurement was performed on GOT, GPT, and ALP using detection kit (Asan Pharmaceutical, Seoul, Korea) by absorbance at 505 nm for GOT and GPT and at 500 nm for ALP, and a standard sample was used as a control.

Total protein volume and albumin were measured by measuring absorbance at nm using a detection kit (Asan Pharmaceutical, Seoul, Korea) and total cholesterol was measured by measuring absorbance at 570 nm using a kit from DoGenBio (Seoul, Korea), and a standard sample was used as a control.

ELISA reader (BioTek, Winooski, VT, USA) was used for the absorbance measurement.

### 5. Changes in Renal Functional Activities

Urea nitrogen was measured using a detection kit (Asan Pharmaceutical, Seoul, Korea) by measuring absorbance at 580 nm, and a standard sample was used as a control.

In addition, creatinine was measured using BioAssay Systems (Hayward, CA, USA), a detection kit, by measuring absorbance at 510 nm, and a standard sample was used as a control.

ELISA reader (BioTek, Winooski, VT, USA) was used for the absorbance measurement.

### 6. Histological Changes in Liver and Kidney

Slides of liver and kidney sections of each laboratory rat were stained with H&E and then photographed with an optical microscope (original magnification, 20×).

### 7. Hepatoprotective Effects of Isosakuranetin in In-Vivo Pharmacokinetic Experiment

Tofacitinib that is being used as a therapeutic agent for rheumarthritis was administered intravenously (10 mg/kg) and orally (20 mg/kg) to rats of the control group (CON), the group with liver cirrhosis induced by DMN (LC), and the group administered with 15 mg/kg of isosakuranetin (ISN) along with DMN (LC-ISN) to identify the hepatoprotective effect of isosakuranetin in the in-vivo pharmacokinetic experiments. Blood collection was performed at 0, 1, 5, 15, 30, 45, 60, 90, 120, 180, 240, 360, 480, 600, and 720 minutes for intravenous administration of tofacitinib, and at 0, 5, 15, 30, 45, 60, 90, 120, 180, 240, 360, 480, 600, and 720 minutes for oral administration to quantify concentrations of tofacitinib in the plasma. Then, urine was collected for 24 hours to analyze the concentration of tofacitinib excreted in urine as well.

### 8. Hepatoprotective Effects of Isosakuranetin in Experiment for In-Vitro Enzyme Activities

Microsomes were isolated from the small intestine and liver of rats in each experimental group, and the enzymatic activity of tofacitinib in microsomal proteins was measured to identify the hepatoprotective effect of isosakuranetin. In the experiment, the concentration of tofacitinib was set at 5, 10, 20, 50, 100, 200, and 500 μM, 1 mg of microsomes and nicotinamide adenine dinucleotide phosphate hydrogen-generating system (Corning Inc., Corning, NY, USA) were blended, and the total volume was adjusted to 1 mL with 0.1 M potassium phosphate buffer (pH 7.4), followed by incubation for 15 minutes at a rate of 50 oscillations per minutes in a water-bath (37° C.). The concentration (Kₘ) of tofacitinib when Vₘₐₓ, the maximum rate of drug metabolism (Vₘₐₓ), was halved was measured by the Lineweaver-Burk plot. The intrinsic clearance (CLᵢₙₜ) of the drug was calculated by dividing Vₘₐₓ by Kₘ.

### 9. Drug Metabolizing Enzyme Recovery Effects of Isosakuranetin

Using microsomes isolated from the small intestine and liver of rats of the CON, LC, and LC-ISN groups, the immunoblot analysis experiment exhibited the drug metabolizing enzyme recovery effect of isosakuranetin. Small intestinal or hepatic microsomal proteins (20-40 mg per lane) were developed in 10% sodium dodecyl sulfate polyacrylamide gel electrophoresis (SDS-PAGE) and then transferred in nitrocellulose membranes. After binding antibodies of P-glycoprotein (P-gp), constitutive androstane receptor (CAR), pregnane X receptor (PXR), β-actin, CYP1A1/2, CYP2B1/2, CYP2C11, CYP2D1, CYP2E1, and CYP3A1/2, protein expression was detected using Image Quant LAS 4000 Mini.

### 10. Statistics

Statistical significance was determined based on Tukey's post-test results after analysis of variance (ANOVA). All experiments were repeated 3 times and expressed as mean±standard deviation (SD). For each of the data,*means p<0.05,**means p<0.01, and means p<0.001.

### Example 1. Changes in Weight by Isosakuranetin Treatment

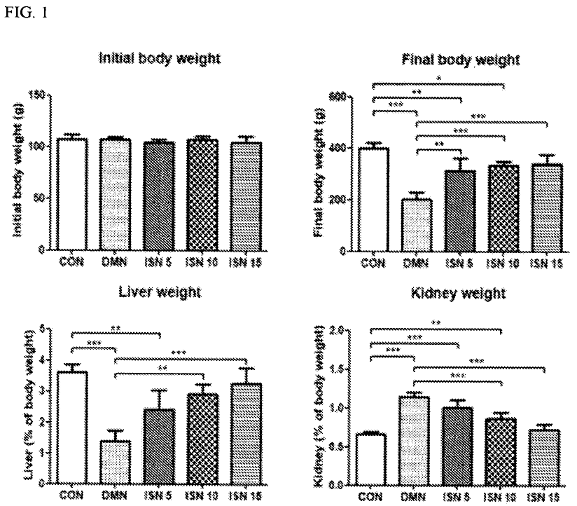

In order to evaluate the hepatoprotective activity by isosakuranetin treatment, the body weight, liver weight, and kidney weight of each rat group were measured, and the results are shown in FIG. 1.

Referring to FIG. 1, it was found that the group treated had significantly less reduction in the body weight than the rats with liver cirrhosis induced by treating DMN (comparison group), and the group treated with 10 and 15 mg/kg of isosakuranetin (ISN 10, ISN 15) showed little difference from the control group (CON).

In addition, rats induced with liver cirrhosis by treating DMN showed a significant decrease in the liver weight by 61.3% compared to the control group and a significant increase in the kidney weight by 72.7% compared to the control group, but the isosakuranetin treated group showed a 33.4% decrease (ISN 5), a 19.6% decrease (ISN 10), and a 9.94% decrease (ISN 15) in the liver weight compared to the control group, respectively, and a 51.7% increase (ISN 5), 29.4% increase (ISN 10) and 9.76% increase (ISN 15) in the kidney weight compared to the control group. Thereby, it can be seen that the tendency that the weight of the liver decreases depending on isosakuranetin is significantly reduced, while the tendency that the weight of the kidney increases is significantly reduced.

### Example 2. Hepatic and Renal Functional Activity by Isosakuranetin Treatment

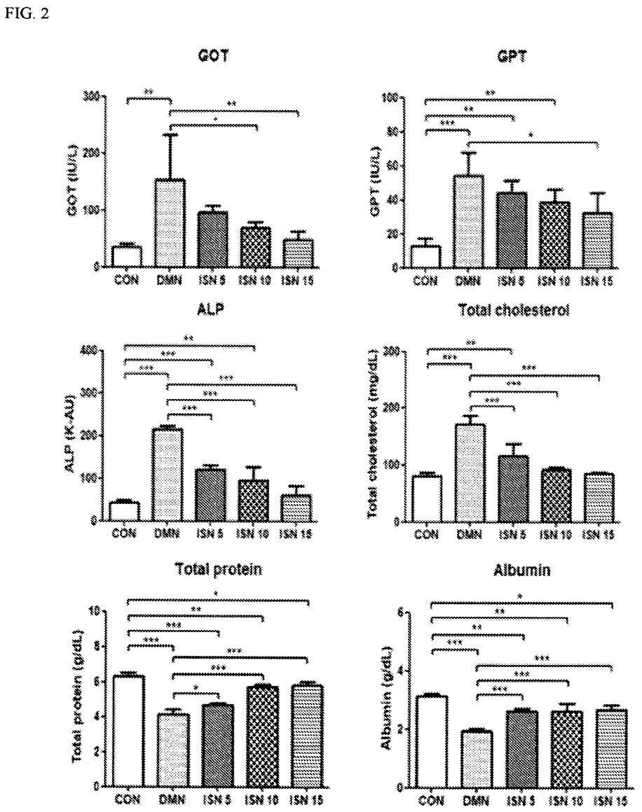

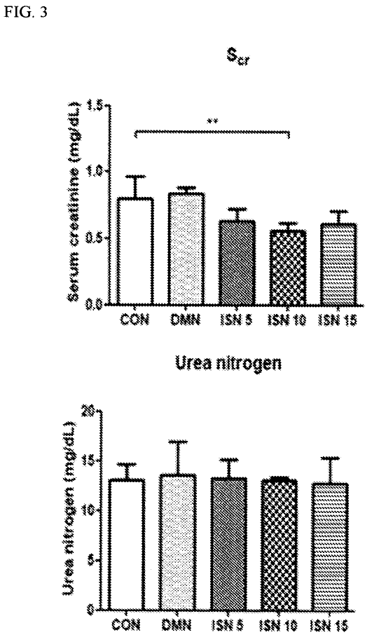

In order to evaluate the hepatoprotective activity and renal protective activity by isosakuranetin treatment, experiments for the hepatic functional and renal functional activity of each laboratory rat were performed, and the results are shown in FIG. 2, FIG. 3, and Table 1.

TABLE 1

| | Control | DMN | ISN 5 | ISN 10 | ISN 15 |
|---|---|---|---|---|---|
| Parameters | (n = 4) | (n = 3) | (n = 4) | (n = 5) | (n = 4) |
| Body weight (g) | | | | | |
| Initial | 108 ± 4.11 | 108 ± 2.08 | 105 ± 2.38 | 108 ± 2.97 | 105 ± 5.91 |
| Final | 402 ± 19.0 | 203 ± 25.5 | 314 ± 48.4 | 336 ± 14.4 | 342 ± 33.6 |
| Plasma | | | | | |
| GOT (IU/L) | 36.1 ± 5.70 | 154 ± 79.8 | 96.6 ± 12.0 | 70.5 ± 10.4 | 48.9 ± 15.2 |
| GPT (IU/L) | 12.9 ± 4.21 | 54.2 ± 13.6 | 44.2 ± 7.19 | 39.0 ± 7.37 | 32.3 ± 11.8 |
| ALP (K-AU) | 43.2 ± 6.88 | 215 ± 8.02 | 121 ± 10.8 | 96.3 ± 31.1 | 60.8 ± 21.7 |
| Total protein | 6.32 ± 0.220 | 4.11 ± 0.347 | 4.66 ± 0.138 | 5.72 ± 0.169 | 5.85 ± 0.190 |
| (g/dL) | | | | | |
| Albumin (g/dL) | 3.15 ± 0.0713 | 1.93 ± 0.0916 | 2.62 ± 0.0980 | 2.63 ± 0.266 | 2.70 ± 0.144 |
| Total | 81.2 ± 5.45 | 172 ± 14.3 | 116 ± 21.5 | 92.3 ± 4.72 | 84.4 ± 3.46 |
| cholesterol | | | | | |
| (mg/dL) | | | | | |
| S_{CR} (mg/dL) | 0.799 ± 0.168 | 0.840 ± 0.0403 | 0.632 ± 0.0946 | 0.562 ± 0.0615 | 0.609 ± 0.103 |
| Urea nitrogen | 13.1 ± 1.56 | 13.6 ± 3.35 | 13.3 ± 1.83 | 13.1 ± 0.276 | 12.8 ± 2.57 |
| (mg/dL) | | | | | |
| Liver weight | 3.62 ± 0.244 | 1.40 ± 0.334 | 2.41 ± 0.622 | 2.91 ± 0.324 | 3.26 ± 0.513 |
| (% of body | | | | | |
| weight) | | | | | |
| Kidney | 0.666 ± 0.0303 | 1.15 ± 0.0608 | 1.01 ± 0.0955 | 0.862 ± 0.0802 | 0.731 ± 0.0657 |
| weight (% of | | | | | |
| body weight) | | | | | |

Referring to FIG. 2 and Table 1, compared to the control group (CON), rats with liver cirrhosis induced by treating DMN (comparison group) had a 327% increase in GOT, a 320% increase in GPT, a 398% increase in ALP, and a 112% increase in the total cholesterol, whereas the group treated with isosakuranetin had 168% (ISN 5), 95.3% (ISN 10), and 35.5% (ISN 15) increases in GOT, respectively, 243% (ISN 5), 202% (ISN 10), and 150% (ISN 15) increases in GPT, respectively, and 180% (ISN 5), 123% (ISN 10), and 40.7% (ISN 15) increases in ALP, respectively, while the total cholesterol had 42.9% (ISN 5), 13.7% (ISN 10), and 3.94% (ISN 15) increases, respectively. In addition, compared to the control group (CON), the rat with liver cirrhosis induced by treating DMN (comparison group) had a 35.0% decrease in the total protein and a 38.7% decrease in albumin, while the group treated with isosakuranetin had 26.3% (ISN 5), 9.49% (ISN 10), and 7.44% (ISN 15) decreases in the total protein, respectively, and 16.8% (ISN 5), 16.5% (ISN 10), and 14.3% (ISN 15) decreases in albumin, respectively.

Thereby, it can be seen that isosakuranetin protects the liver and suppresses liver cirrhosis in a concentration-dependent manner.

Referring to FIG. 3 and Table 1, compared to the control group (CON), the rats with liver cirrhosis induced by treating DMN (comparison group) did not show a significant difference between creatinine (S_{CR}) and urea nitrogen. In addition, the group treated with isosakuranetin had 20.9% (ISN 5), 29.7% (ISN 10), and 23.8% (ISN 15) increases in creatinine (S_{CR}), respectively, with no significant difference in urea nitrogen.

### Example 3. Histological Changes by Isosakuranetin Treatment

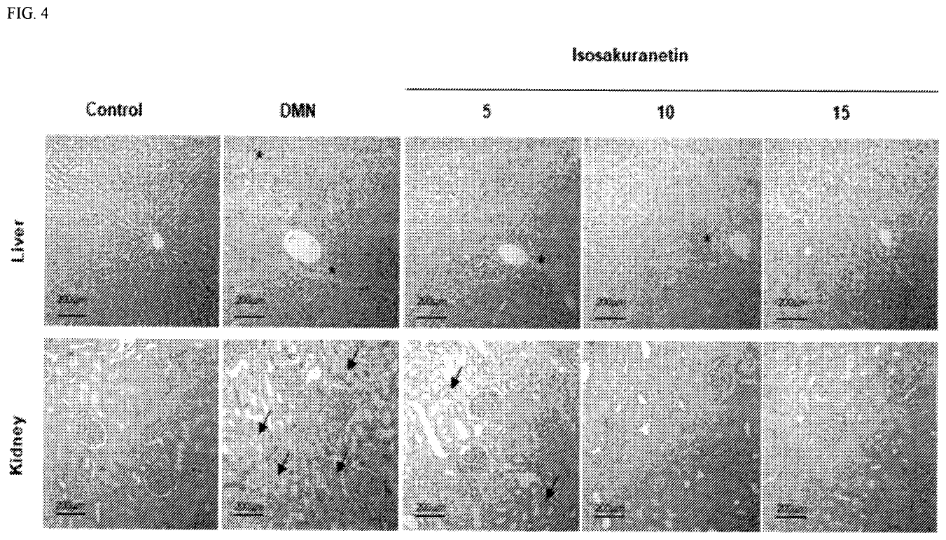

The hepatoprotective activity and renal protective activity by isosakuranetin treatment were photographed under an optical microscope (original magnification, 20×), and the results are shown in FIG. 4. The asterisk in FIG. 4 indicates tissue damage, including necrosis and fibrosis, while the arrows indicate cell death and tissue damage.

Referring to FIG. 4, rats with liver cirrhosis induced by treating DMN (comparison group) showed severe morphological changes including inflammation and necrosis in the liver and kidney compared to the control group (CON). On the other hand, the isosakuranetin treated groups, especially ISN 10 and ISN 15 groups, showed remarkable amelioration and protection from liver damage accompanied with necrosis and fibrosis and kidney tissue damage as well as mass cell death.

### Example 4. Hepatoprotective Effects of Isosakuranetin in In-Vivo Pharmacokinetic Experiments

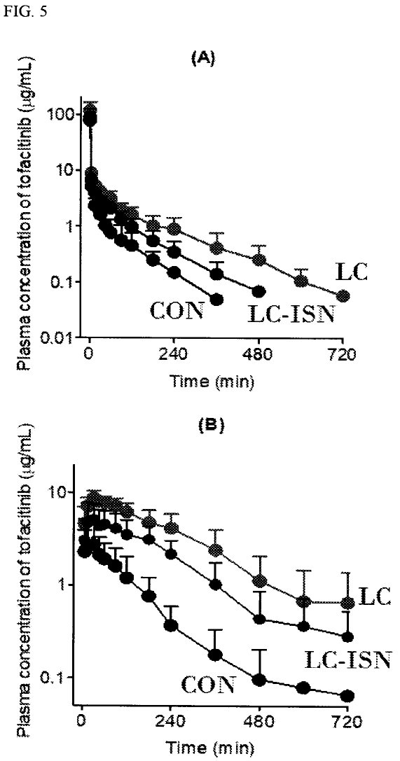

The mean plasma concentration curve and corresponding pharmacokinetic parameters after intravenous (10 mg/kg) and oral (20 mg/kg) administration of tofacitinib in CON, LC, and LC-ISN rats are shown in FIG. 5, Table 2, and Table 3, respectively.

TABLE 2

| | CON | LC | LC-ISN |
|---|---|---|---|
| Parameters | (n = 5) | (n = 6) | (n = 7) |
| Body weight (g) ^{a} | 430 ± 20.0 | 249 ± 67.6 | 309 ± 30.6 |
| Terminal half-life | 35.4 ± 19.2 | 113 ± 43.7 | 58.2 ± 16.8 |
| (min) ^{b} | | | |
| AUC | 440 ± 211 | 1135 ± 391 | 737 ± 107 |
| (μg · min/mL) ^{b} | | | |
| CL (mL/min/kg) ^{c} | 24.1 ± 6.27 | 11.0 ± 2.06 | 13.8 ± 1.73 |
| CL_{R} (mL/min/kg) | 3.73 ± 1.09 | 4.65 ± 2.74 | 4.10 ± 1.64 |
| CL_{NR} | 21.1 ± 4.57 | 6.35 ± 1.27 | 9.68 ± 2.40 |
| (mL/min/kg) ^{a} | | | |
| Vₛₛ (mL/kg) | 429 ± 253 | 798 ± 177 | 800 ± 326 |
| Ae_{0-24 h} | 13.9 ± 2.28 | 40.2 ± 18.9 | 30.1 ± 12.3 |
| (% of dose) ^{d} | | | |
| GI_{24 h} (% of dose) ^{e} | 0.164 ± 0.102 | 2.07 ± 2.12 | 0.166 ± 0.135 |

CON, control;
LC, liver cirrhosis induced by N-dimethylnitrosamine;
LC-ISN, LC treated with isosakuranetin.
^{a} CON is significantly different from LC (p < 0.001) and LC-ISN (p < 0.001).
^{b} CON is significantly different from LC (p < 0.01) and LC-ISN is significantly different from LC (p < 0.05).
^{c} CON is significantly different from LC (p < 0.001) and LC-ISN (p < 0.01).
^{d} CON is significantly different from LC (p < 0.05).
^{e} LC-ISN is significantly different from LC (p < 0.05).

TABLE 3

| | CON | LC | LC-ISN 15 |
|---|---|---|---|
| Parameters | (n = 6) | (n = 7) | (n = 7) |
| Body | 386 ± 28.2 | 222 ± 36.6 | 255 ± 38.6 |
| weight (g) ^{a} | | | |
| AUC | 384 ± 122 | 2031 ± 500 | 1240 ± 128 |
| (μg · min/mL) ^{b} | | | |
| Cₘₐₓ (μg/mL) ^{c} | 3.53 ± 1.46 | 9.10 ± 1.26 | 6.08 ± 1.99 |
| Tₘₐₓ (min) | 50.0 ± 44.2 | 55.7 ± 36.4 | 85.7 ± 79.7 |
| CL_{R} | 6.98 ± 2.99 | 3.62 ± 1.78 | 5.71 ± 2.47 |
| (mL/min/kg) | | | |
| Ae_{0-24 h} | 11.8 ± 4.08 | 35.8 ± 16.9 | 35.9 ± 16.8 |
| (% of dose) ^{d} | | | |
| GI_{24 h} | 0.106 ± 0.101 | 1.73 ± 2.04 | 0.340 ± 0.288 |
| (% of dose) | | | |
| F (%) | 43.6 | 89.5 | 84.1 |

CON, control;
LC, liver cirrhosis induced by N-dimethylnitrosamine;
LC-ISN, LC treated with isosakuranetin.
^{a} CON differs significantly from LC (p < 0.001) and LC-ISN (p < 0.001).
^{b} CON differs significantly from LC (p < 0.001) and LC-ISN (p < 0.001), and LC-ISN differs significantly from LC (p < 0.001).
^{c} CON differs significantly from LC (p < 0.001) and LC-ISN (p < 0.05), and LC-ISN differs significantly from LC (p < 0.01).
^{d} CON differs significantly from LC (p < 0.05) and LC-ISN (p < 0.05).

Table 2 shows the pharmacokinetic parameters of tofacitinib after intravenous administration of 10 mg/kg of tofacitinib to CON, LC and LC-ISN rats, and Table 3 shows the pharmacokinetic parameters of tofacitinib after oral administration of 20 mg/kg of tofacitinib to CON, LC and LC-ISN rats. Referring to Table 2, after intravenous administration of 10 mg/kg of tofacitinib, the area under the plasma concentration-time curve from time 0 to time infinity (AUC) of tofacitinib increased significantly by 158% in the LC group compared to the CON group. This appears to be due reduction in the total body clearance (CL) and nonrenal clearance (CL_{NR}) by 54.4% and 69.9%, respectively in the LC group, compared to the CON group. The LC-ISN group had 35.1% reduction in AUC of tofacitinib compared to the LC group. Showing a hepatoprotective effect by isosakuranetin (ISN) treatment in the LC-ISN group, it was determined that the plasma concentration was close to the CON group.

Referring to Table 3, after oral administration of 20 mg/kg of tofacitinib, AUC of tofacitinib in the LC group increased significantly by 429% compared to the CON group. In the LC-ISN group, the AUC of tofacitinib was significantly reduced by 38.9% compared to the LC group. This also showed a hepatoprotective effect by administration of isosakuranetin (ISN), showing that the plasma concentration of tofacitinib was close to the CON group.

### Example 5. Hepatoprotective Effects of Isosakuranetin in Experiments for In-Vitro Enzyme Activity

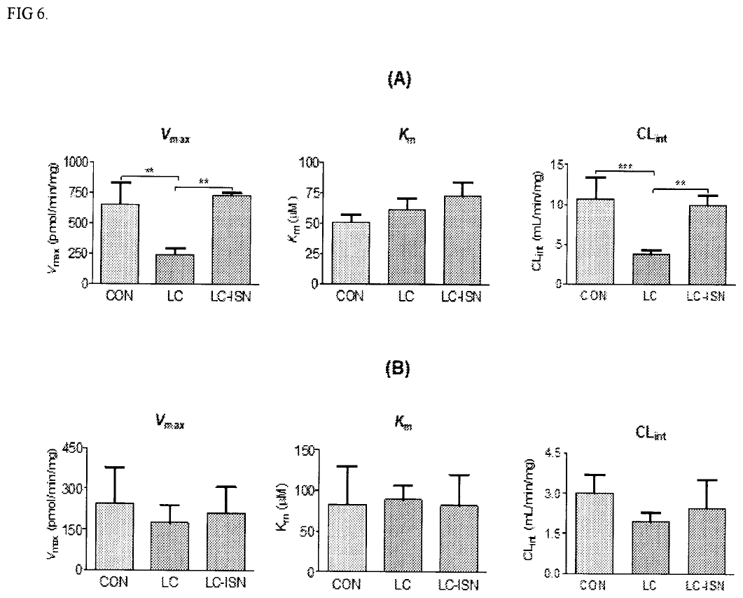

The values of Vₘₐₓ, Kₘ, and CLᵢₙₜ measured by the in-vitro experiment for enzyme activity using small intestinal and hepatic microsomes of the CON, LC, and LC-ISN groups are shown in FIG. 6.

Referring to FIG. 6, in the hepatic microsome, the Vₘₐₓ of tofacitinib decreased significantly by 63.4% in the LC group compared to the CON group, but the LC-ISN group showed a significant increase by 204% compared to the LC group and recovered to the level of CON group, showing no significant difference. No significant difference in Kₘ observed in the three groups. Therefore, in hepatic microsomes, the CLᵢₙₜ of tofacitinib had a significant decrease by 64.9% in the LC group compared to the CON group, but the LC-ISN group had a significant increase by 163% compared to the LC group and recovered to 92.2% of the CON group. On the other hand, in the small intestinal microsome, there was a tendency that CLᵢₙₜ in the LC group decreased compared to the CON group and the LC-ISN group, but there was no statistically significant difference.

### Example 6. Drug Metabolizing Enzyme Recovery Effects by Isosakuranetin

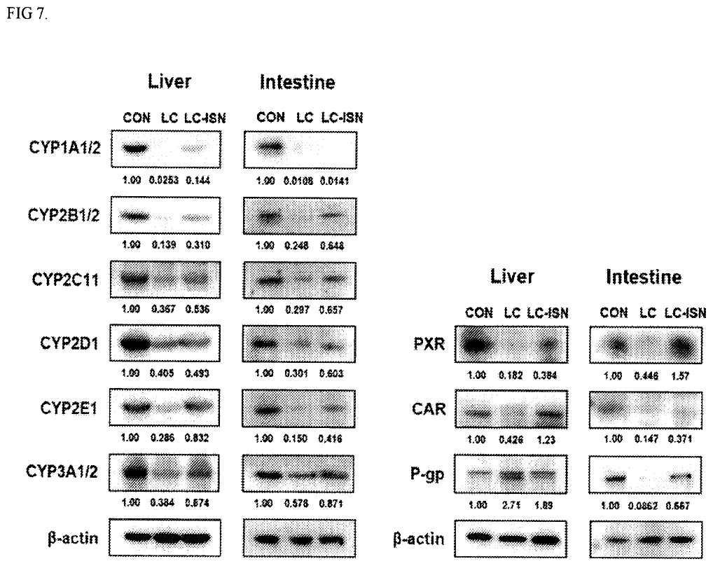

Protein expression of CYP isoforms, P-gp, CAR, and PXR in small intestinal and hepatic microsomes of rats of the CON, LC, and LC-ISN groups is shown in FIG. 7.

As shown in FIG. 7, the expression of CYP enzymes in the hepatic and small intestinal microsomes in the LC group was significantly decreased compared to the CON group, but recovered partially in the LC-ISN group, showing a tendency close to the CON group. In particular, as a result of examining the expression of CYP2C11 and CYP3A1/2 which are the main metabolizing enzymes of tofacitinib, CYP2C11 decreased by 61.6% and 42.2% respectively in the hepatic and small intestinal microsomes in the LC group compared to the CON group, but increased by 67.4% and 87.1% of in the hepatic and small intestinal microsomes, respectively, in the LC-ISN group compared to the LC group. PXR and CAR, transcription factors that regulate the CYP enzyme, also showed a similar pattern as the changes in CYP expression. In the case of P-gp, the expression patterns in the small intestinal and hepatic microsomes in the LC group are different from those of CYP isoforms, PXR, and CAR, but the tendency for the LC-ISN group to approach that of the CON group was similar to that of CYP isoforms, PXR, and CAR, suggesting that this was a hepatoprotective effect caused by isosakuranetin (ISN).

As described above, specific parts of the present disclosure have been described in detail, and for those skilled in the art, it is clear that the specific descriptions are merely preferred example embodiments and the scope of the present invention is not limited thereby. In other words, the substantial scope of the present invention is defined by the appended claims and their equivalents.

1. A method of preventing or treating liver cirrhosis, comprising:
administering a pharmaceutical composition comprising isosakuranetin or a pharmaceutically acceptable salt thereof as an active ingredient to a subject.

2. The method of claim 1, wherein the isosakuranetin inhibits tissue damage including necrosis and fibrosis in liver and inhibits tissue damage accompanied by massive cell death in kidney.

3. The method of claim 1, wherein the isosakuranetin restores drug metabolizing enzymes reduced by liver cirrhosis.

4. The method of claim 1, wherein the pharmaceutical composition comprises the isosakuranetin at a concentration of 3 to 20 mM.

5. The method of claim 1, wherein the isosakuranetin is administered in an amount of 1 to 20 mg/kg of body weight.

6. A method preventing or ameliorating liver cirrhosis, comprising:
administering a health functional food composition comprising isosakuranetin as an active ingredient to a subject.

7. The method of claim 6, wherein the isosakuranetin inhibits tissue damage including necrosis and fibrosis in liver and inhibits tissue damage accompanied by massive cell death in kidney.

8. A method of helping liver health, comprising:
administering a health functional food composition comprising isosakuranetin as an active ingredient to a subject.

9. The method of claim 8, wherein the health functional food composition is powder, granule, tablet, capsule, syrup, or beverage.
